(19) United States
(12) Patent Application Publication
(10) Pub. No.: US 2022/0248976 A1
(43) Pub. Date: Aug. 11, 2022

(54) PROSTATE CANCER DETECTING MEDICAL DEVICES

(71) Applicant: NOVASCAN, INC., Milwaukee, WI (US)

(72) Inventors: Les BOGDANOWICZ, Park Ridge, IL (US); Donato M. CERES, Chicago, IL (US); Craig Quinn DAVIS, Chicago, IL (US); Daniel Steven GEHRKE, Chicago, IL (US); Alexander GRYCUK, Mt. Prospect, IL (US)

(21) Appl. No.: 17/732,390

(22) Filed: Apr. 28, 2022

Related U.S. Application Data
(63) Continuation-in-part of application No. 17/695,748, filed on Mar. 15, 2022.
Application No. 17/695,748 is a continuation-in-part of application No. 17/397,896, filed on Aug. 9, 2021.
Application No. 17/695,748 is a continuation-in-part of application No. 17/412,973, filed on Aug. 26, 2021.
(60) Provisional application No. 63/142,242, filed on Jan. 27, 2021.
(60) Provisional application No. 63/142,247, filed on Jan. 27, 2021.
(60) Provisional application No. 63/142,254, filed on Jan. 27, 2021.
(60) Provisional application No. 63/142,260, filed on Jan. 27, 2021.
(60) Provisional application No. 63/142,242, filed on Jan. 27, 2021.
(60) Provisional application No. 63/142,247, filed on Jan. 27, 2021.
(60) Provisional application No. 63/142,254, filed on Jan. 27, 2021.
(60) Provisional application No. 63/142,260, filed on Jan. 27, 2021.

(51) Int. Cl. A61B 5/0537; A61B 5/0538; A61B 5/00; A61B 90/00; A61B 5/107

(52) U.S. Cl. CPC A61B 5/0537; A61B 5/0538; A61B 5/0002; A61B 5/6853; A61B 90/06; A61B 2090/062; A61B 5/742; A61B 5/7405; A61B 5/7221; A61B 5/4381; A61B 5/1076

(57) ABSTRACT

In various embodiments, a medical device comprises a urethral catheter including a distal portion two or more electrodes disposed on the distal portion; an impedance bridge coupled to the two or more electrodes; and a processor coupled to the impedance bridge. In various embodiments, a computer-implemented method for determining one or more tissue types at a location associated with a distal portion of a urethral catheter comprises recording, at one or more frequencies, one or more impedance measurements, wherein each impedance measurement is associated with two or more electrodes disposed on the distal portion of the urethral catheter; comparing the one or more impedance measurements to one or more characteristic impedances associated with one or more tissue types; and determining, based on the one or more impedance measurements and the one or more characteristic impedances, one or more tissue types at a location associated with the distal portion.

### CROSS-REFERENCES TO RELATED APPLICATIONS

The present application is a continuation-in-part of the U.S. patent application titled, “MEDICAL DEVICES CONFIGURED WITH NEEDLE ELECTRODES,” filed on Mar. 15, 2022, and having Ser. No. 17/695,748, which is a continuation-in-part of the U.S. patent application titled, “IMPEDANCE-CALIBRATED DIAGNOSTIC MEDICAL DEVICES,” filed on Aug. 9, 2021, and having Ser. No. 17/397,896, which claims the benefit of U.S. Provisional Patent Application No. 63/142,242, filed Jan. 27, 2021; U.S. Provisional Patent Application No. 63/142,247, filed Jan. 27, 2021; U.S. Provisional Patent Application No. 63/142,254, filed Jan. 27, 2021; and U.S. Provisional Patent Application No. 63/142,260, filed Jan. 27, 2021, and which is also a continuation-in-part of the U.S. patent application titled, “TECHNIQUES FOR CONTROLLING MEDICAL DEVICE TOOLS,” filed on Aug. 26, 2021, and having Ser. No. 17/412,973, which claims the benefit of U.S. Provisional Patent Application No. 63/142,242, filed Jan. 27, 2021; U.S. Provisional Patent Application No. 63/142,247, filed Jan. 27, 2021; U.S. Provisional Patent Application No. 63/142,254, filed Jan. 27, 2021; and U.S. Provisional Patent Application No. 63/142,260, filed Jan. 27, 2021. The subject matter of these related applications is hereby incorporated herein by reference.

### BACKGROUND

### Field of the Various Embodiments

Embodiments of the present disclosure relate generally to electronics and medical diagnostic technology and, more specifically, to prostate cancer detecting medical devices.

### Description of the Related Art

Prostate cancer is often detected by a digital rectal examination (DRE), in which a healthcare professional determines the firmness of the prostate. A hard or inconsistent firmness prostate can indicate cancer, while a soft and consistent firmness can indicate an absence of cancer.

One drawback of DRE testing for prostate cancer is that this type of testing oftentimes is inaccurate. Among other things, DRE requires a subjective measurement of firmness, and different healthcare professionals can reach different conclusions based on examinations of the same prostate. False positive DRE test results can occur due to the subjective nature of the examination as well as any firmness caused by noncancerous conditions. False positive test results can result in unnecessary medical procedures that can damage the prostate be painful and expensive as well. Similarly, false negative DRE test results can occur when a patient has cancer but the patient's prostate is not firm or inconsistent. False negative test results can result in a failure to detect and treat prostate cancer at an early stage, where treatment options at later stages can be more invasive and can present poorer prognoses. Another drawback of DRE testing for prostate cancer is that this type of testing sometimes can present inconclusive results, which can require additional testing and can cause anxiety for the patient.

In another approach, prostate cancer can be detected by measuring a level of prostate-specific antigen (PSA) in a blood sample of the patient. Elevated levels of PSA as compared with a baseline level can indicate cancer.

One drawback of testing PSA levels for prostate cancer is that this type of testing also is oftentimes inaccurate. Elevated PSA levels can occur due to noncancerous conditions, such as prostate inflammation or prostate enlargement, leading to false positive test results. False positive PSA tests can result in unnecessary medical procedures that can damage the prostate and be painful and expensive as well. Also, false negative PSA test results can occur when a patient has cancer but the cancer does not cause an increase in PSA levels. Again, false negative tests can result in a failure to detect and treat prostate cancer at an early stage, where treatment options at later stages can be more invasive and can present poorer prognoses. As with DRE testing, another drawback of PSA testing for prostate cancer is that this type of testing can present inconclusive results, which can require additional testing and can cause anxiety for the patient.

As the foregoing illustrates, what is needed in the art are more effective prostate cancer detecting medical devices.

### SUMMARY

Embodiments are disclosed for medical devices. In various embodiments, the medical device includes a urethral catheter including a distal portion and two or more electrodes disposed on the distal portion; an impedance bridge coupled to the two or more electrodes; and a processor coupled to the impedance bridge.

In various embodiments, a medical device comprises a urethral catheter including a distal portion two or more electrodes disposed on the distal portion; an impedance bridge coupled to the two or more electrodes; and a processor coupled to the impedance bridge.

In various embodiments, a computer-implemented method for determining one or more tissue types at a location associated with a distal portion of a urethral catheter comprises recording, at one or more frequencies, one or more impedance measurements, wherein each impedance measurement is associated with two or more electrodes disposed on the distal portion of the urethral catheter; comparing the one or more impedance measurements to one or more characteristic impedances associated with one or more tissue types; and determining, based on the one or more impedance measurements and the one or more characteristic impedances, one or more tissue types at a location associated with the distal portion.

At least one technical advantage of the disclosed medical device relative to the prior art is that the disclosed medical device is able to automatically and accurately determine the tissue type at a location associated with the distal portion of the urethral catheter. For example, the disclosed urethral catheter can accurately determine whether the tissue type associated with a prostate is a cancerous tissue type or a non-cancerous tissue type. Determining the tissue type associated with a prostate with high accuracy can advantageously reduce the incidence of false positive testing outcomes and reduce the occurrence of unnecessary medical procedures. Determining the tissue type associated with a prostate with high accuracy also can advantageously reduce the incidence of false negative testing outcomes and enable effective cancer treatment at early stages and with better prognoses. In addition, the disclosed medical device can automatically and accurately determine whether the tissue type at a location associated with the distal portion of the urethral catheter matches a tissue type associated with a prostate. Such determinations can improve overall confidence in the test results. Likewise, automatically and accurately determining that the tissue type at a location associated distal portion of the urethral catheter does not match the tissue type associated with a prostate can reduce incorrect test results and give a healthcare provider an opportunity to reposition the urethral catheter. These technical advantages provide one or more technological advancements over prior art designs and approaches.

### BRIEF DESCRIPTION OF THE DRAWINGS

FIG. 1 illustrates a medical device configured to implement one or more aspects of the various embodiments;

FIG. 2 is another illustration of the medical device of FIG. 1, according to various embodiments;

FIG. 3 is a more detailed illustration of a distal portion of the medical device of FIG. 2, according to various embodiments;

FIG. 4 is a more detailed illustration of another distal portion of the medical device of FIG. 2, according to various embodiments;

FIGS. 5A-5B are a more detailed illustration of yet another distal portion of the medical device of FIG. 2, according to various embodiments;

FIG. 6 is a more detailed illustration of the external electrical components of the medical device of FIG. 2, according to various embodiments;

FIG. 7 is a more detailed illustration of the medical device of FIG. 2, according to various other embodiments; and

FIG. 8 is a flow diagram of method steps for determining one or more tissue types at a location associated with a distal portion of a urethral catheter, according to various embodiments.

### DETAILED DESCRIPTION

In the following description, numerous specific details are set forth to provide a more thorough understanding of the various embodiments. However, in the range of embodiments of the concepts includes some embodiments omitting one or more of these specific details.

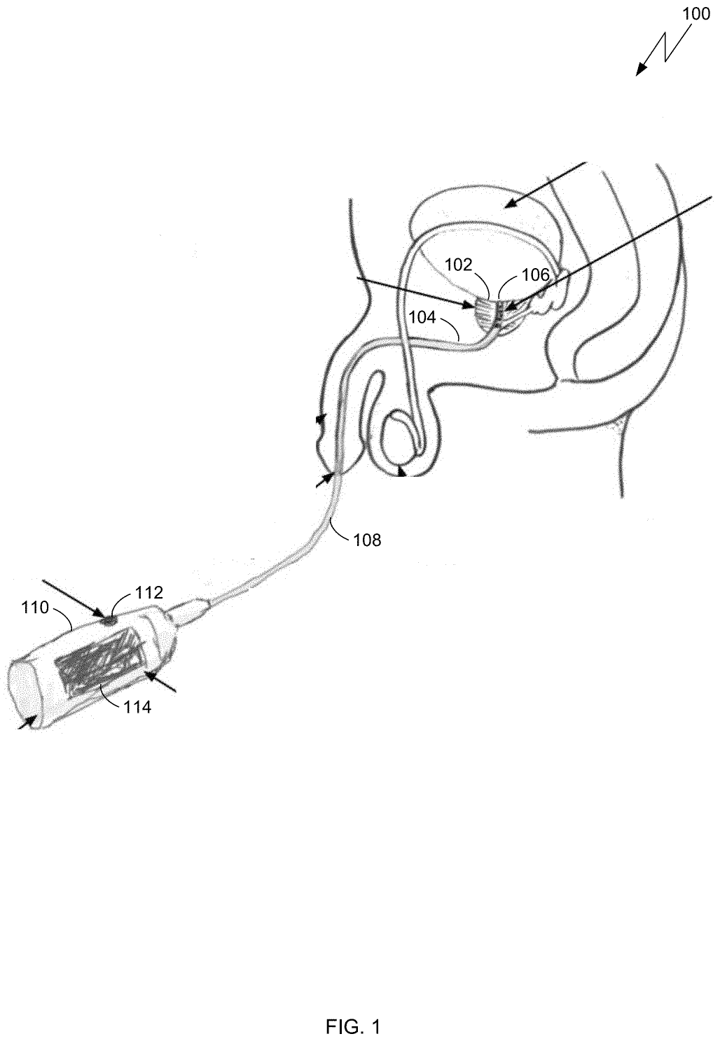

FIG. 1 illustrates a medical device 100, according to various embodiments. As shown, the medical device 100 includes, without limitation, a urethral catheter 108 and external electrical components 110. The urethral catheter 108 includes, without limitation, a distal portion 106. The external electrical components 110 include, without limitation, an activation button 112 and a display 114.

The urethral catheter 108 is inserted into a urethra 104 of a patient. The distal portion 106 of the urethral catheter 108 is positioned within the urethra 104 at the location of the prostate 102 of the patient. The external electrical components 110 generate current at various frequencies. Wires within the urethral catheter 108 conduct the current between the external electrical components 110 and two or more electrodes disposed on the distal portion 106. The external electrical components 110 include a processor that couples to the two or more electrodes disposed on the distal portion 106. The processor of the external electrical components 110 measures the impedance of current conducted through tissue between at least two of the two or more electrodes. As described in greater detail below, the medical device 100 determines, based on the impedance measurements, one or more tissue types at the location associated with the distal portion 106.

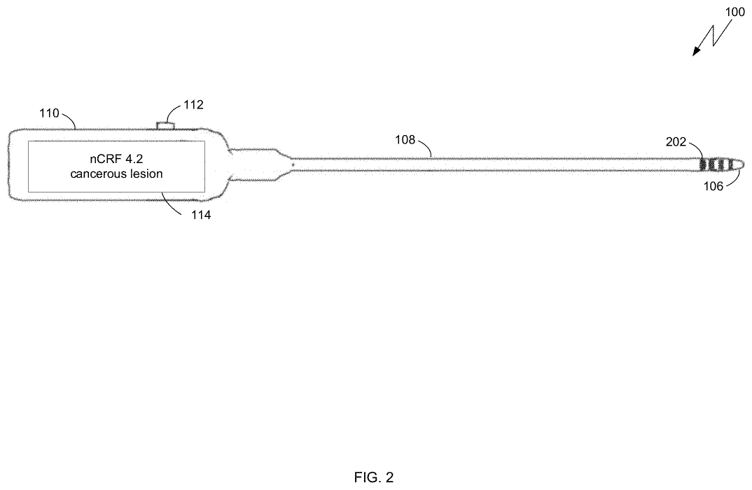

FIG. 2 is another illustration of the medical device of FIG. 1, according to various embodiments. As shown, the medical device 100 includes, without limitation, a urethral catheter 108 and external electrical components 110. The urethral catheter 108 includes, without limitation, a distal portion 106 and two or more electrodes 202 disposed on the distal portion 106. The external electrical components 110 include, without limitation, an activation button 112 and a display 114.

As previously discussed, the external electrical components 110 include a processor that couples to the two or more electrodes 202 disposed on the distal portion 106. In response to an activation associated with the activation button 112, the processor of the external electrical components 110 measures the impedance of current conducted through tissue between at least two of the two or more electrodes 202 and records, at one or more frequencies, one or more impedance measurements.

Based on the one or more impedance measurements and the one or more characteristic impedances of various tissue types, the processor determines one or more tissue types at the location associated with the distal portion 106. For example and without limitation, based on the impedance measurements, the tissue type can indicate whether tissue at the location associated with the distal portion 106 is cancer tissue or non-cancer tissue. For example and without limitation, based on the impedance measurements, the tissue type can indicate whether tissue at the location associated with the distal portion 106 is prostate tissue or non-prostate tissue. As shown, the medical device 100 displays the tissue type on the display 114. In some embodiments and as shown, the medical device 100 also displays one or more properties associated with the impedance measurements, such as a normalized Cole relaxation function (“nCRF”) measurement.

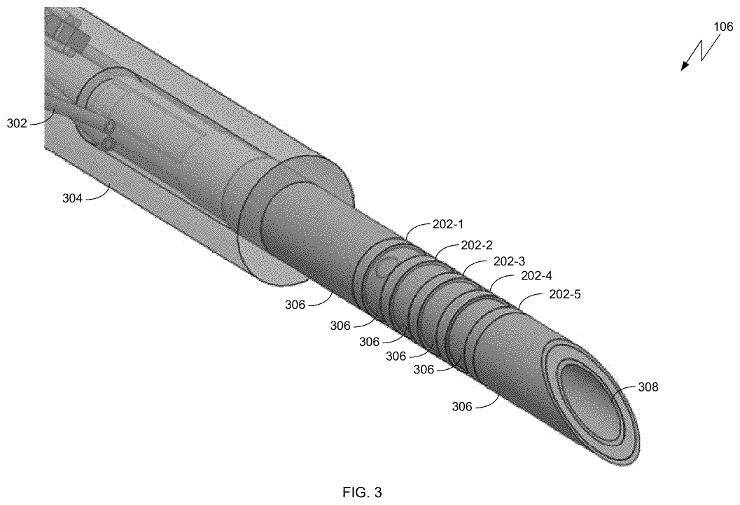

FIG. 3 is a more detailed illustration of a distal portion 106 of the medical device of FIG. 2, according to other various embodiments. As shown, the distal portion 106 includes, without limitation, a set of electrodes 202-1 to 202-5, a conduit 304, electrically insulating material 306, and a tip 308. As shown, the conduit 304 includes wires 302.

As shown, each of the electrodes 202 encircles the distal portion 106 at a respective location along a length of the distal portion 106. For example and without limitation, the first electrode 202-1 encircles the distal portion 106 at a first location near the tip 308, and the second electrode 202-2 encircles the distal portion 106 at a second location that is further from the tip 308. As shown, the electrodes 202 are disposed on top of a layer of electrically insulating material 306. The electrically insulating material 306 is also located between adjacent pairs of electrodes 202. As shown, the electrically insulating material 306 encircles the distal portion 106 between each pair of adjacent electrodes 202. The electrically insulating material 306 can reduce contact and short circuits between adjacent electrodes 202, which could reduce the accuracy of the impedance measurements.

When the distal portion 106 is positioned at a location in the urethra of a patient, the external electrical components 110 can record, at one or more frequencies, one or more impedance measurements, wherein each impedance measurement is associated with two or more of the electrodes 202. In various embodiments, the external electrical components 110 are selectively coupled to two or more selected electrodes 202 of the two or more electrodes 202. While the external electrical components 110 are selectively coupled to a first subset of the two or more electrodes 202 (e.g., the first electrode 202-1 and the second electrode 202-2), the medical device can record a first subset of impedance measurements of tissue between or contacting the first subset of the two or more electrodes 202. While the external electrical components 110 are selectively coupled to a second subset of the two or more electrodes 202 (e.g., the third electrode 202-3 and the fourth electrode 202-4), the medical device can record a second subset of impedance measurements of tissue between or contacting the second subset of the two or more electrodes 202. Based on the first subset and second subset of impedance measurements, the medical device can determine a first tissue type between the first electrode 202-1 and the second electrode 202-2 and a second tissue type between the third electrode 202-3 and the fourth electrode 202-4. For example and without limitation, the first tissue type based on the first subset of impedance measurements can indicate that the tissue near the tip 308 is a cancer tissue type, and the second tissue type based on the second impedance measurement can indicate that the tissue further from the tip 308 is a non-cancer tissue type. As another example and without limitation, the first tissue type based on the first impedance measurement can indicate that the tissue near the tip 308 is a prostate tissue type, and the second tissue type based on the second impedance measurement can indicate that the tissue further from the tip 308 is a non-prostate tissue type. Based on the first and second tissue types, the external electrical components 110 can determine the tissue type of the prostate 102 and/or whether the distal portion 106 of the urethral catheter 108 is positioned at the location of the prostate 102.

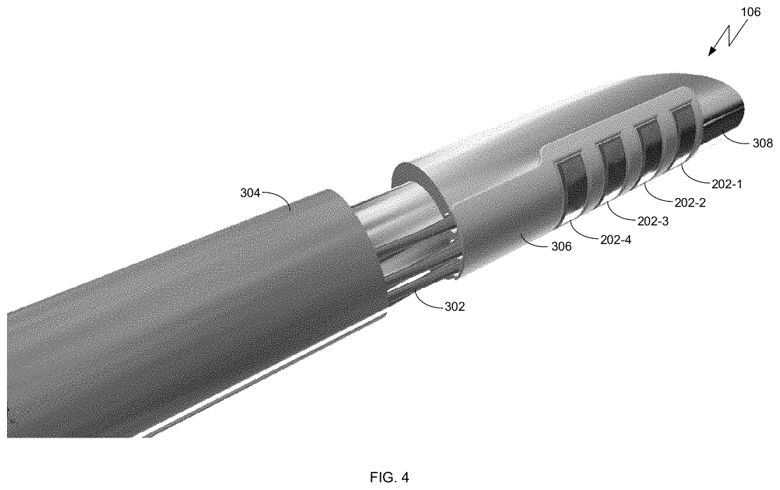

FIG. 4 is a more detailed illustration of another distal portion 106 of the medical device of FIG. 2, according to other various embodiments. As shown, the distal portion 106 includes, without limitation, a set of electrodes 202-1 to 202-4, a conduit 304, electrically insulating material 306, and a tip 308. As shown, the conduit 304 includes wires 302.

In various embodiments, the electrically insulating material 306 is located between adjacent pairs of electrodes 202 along the length of the distal portion 106. As shown, the electrically insulating material 306 includes a set of carve-outs, and each of the two or more electrodes 202 is located within one of the carve-outs of the electrically insulating material 306. The electrically insulating material 306 can reduce contact and short circuits between adjacent electrodes 202, which could reduce the accuracy of the impedance measurements.

In various embodiments, the external electrical components 110 are selectively coupled to two or more selected electrodes 202 of the two or more electrodes 202. While the external electrical components 110 are selectively coupled to a first subset of the two or more electrodes 202 (e.g., the first electrode 202-1 and the second electrode 202-2), the medical device can record a first subset of impedance measurements of tissue between or contacting the first subset of the two or more electrodes 202. While the external electrical components 110 are selectively coupled to a second subset of the two or more electrodes 202 (e.g., the third electrode 202-3 and the fourth electrode 202-4), the medical device can record a second subset of impedance measurements of tissue between or contacting the second subset of the two or more electrodes 202. Based on the first subset and second subset of impedance measurements, the medical device can determine a first tissue type between the first electrode 202-1 and the second electrode 202-2 and a second tissue type between the third electrode 202-3 and the fourth electrode 202-4. For example and without limitation, the first tissue type based on the first impedance measurement can indicate that the tissue near the tip 308 is a cancer tissue type, and the second tissue type based on the second impedance measurement can indicate that the tissue further from the tip 308 is a non-cancer tissue type. As another example and without limitation, the first tissue type based on the first impedance measurement can indicate that the tissue near the tip 308 is a prostate tissue type, and the second tissue type based on the second impedance measurement can indicate that the tissue further from the tip 308 is a non-prostate tissue type. Based on the first and second tissue types, the external electrical components 110 can determine the tissue type of the prostate 102 and/or whether the distal portion 106 of the urethral catheter 108 is positioned at the location of the prostate 102.

While not shown, in various embodiments, one or more of the two or more electrodes 202 is located on a first side of the distal portion 106, and at least another one or more of the two or more electrodes 202 is located on a second side of the distal portion 106. For example and without limitation, the first electrode 202-1 and the second electrode 202-2 could be located on a left side of the distal portion 106. The external electrical components 110 could selectably couple to a first subset of the two or more electrodes 202 (e.g., the first electrode 202-1 and the second electrode 202-2) to record a first subset of impedance measurements. Based on the first subset of impedance measurements, the external electrical components 110 could determine a tissue type on the left side of the distal portion 106. Also, the third electrode 202-3 and the fourth electrode 202-4 could be located on a right side of the distal portion 106. The external electrical components 110 could selectably couple to a second subset of the two or more electrodes 202 (e.g., the third electrode 202-3 and the fourth electrode 202-4) to record a second subset of impedance measurements. Based on the second subset of impedance measurements, the external electrical components 110 could determine a tissue type on the right side of the distal portion 106. Based on the first and second subsets of impedance measurements, the external electrical components 110 could determine the tissue types on different sides of the distal portion 106. For example and without limitation, the external electrical components 110 could determine that the tissue on a left side of the prostate 102 is a cancer tissue type, and that the tissue on a right side of the prostate 102 is a non-cancer tissue type.

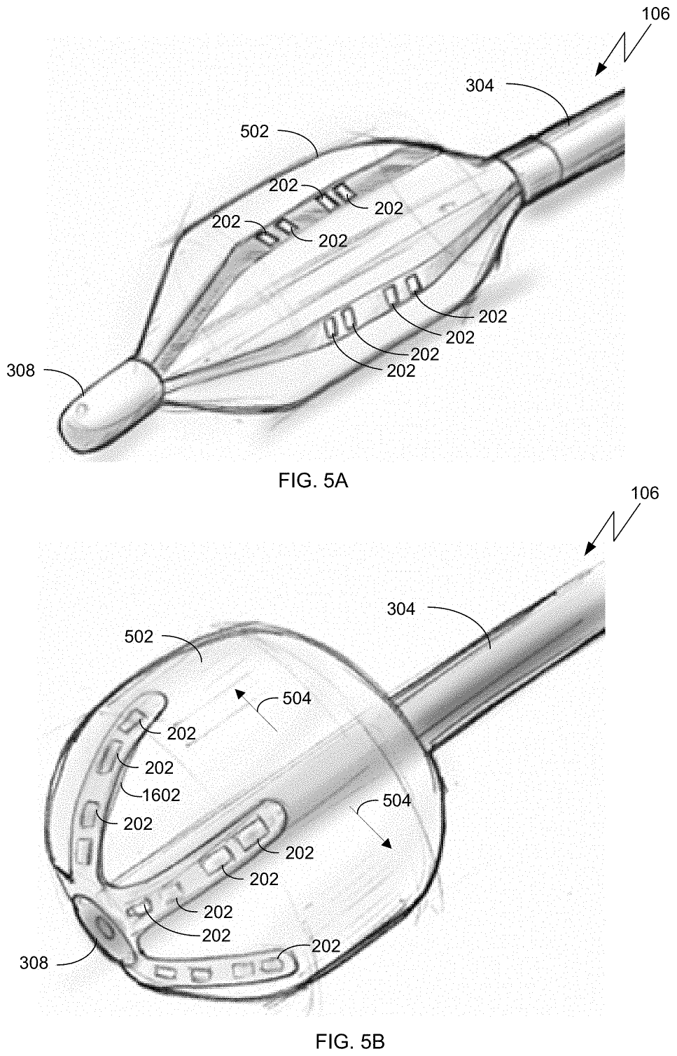

FIGS. 5A-5B are a more detailed illustration of yet another distal portion 106 of the medical device of FIG. 2, according to various embodiments. As shown, the distal portion 106 includes electrodes 202, a conduit 304, a tip 308, and a balloon 502.

As shown in FIG. 5A, the balloon 502 is in a collapsed configuration. The external electrical components 110 can record a first set impedance measurements of two or more electrodes 202 while the balloon 502 is in the collapsed configuration to determine impedance measurements and tissue types of tissue at the location associated with the distal portion 106. For example and without limitation, the external electrical components 110 can record a first subset of impedance measurements between two or more electrodes 202 at a first position along a length of the distal portion 106 and a second subset of impedance measurements between two or more electrodes 202 at a second position along the length of the distal portion 106. As another example and without limitation, the external electrical components 110 can record a first subset of impedance measurements between two or more electrodes 202 disposed on a left side of the distal portion 106 and a second subset of impedance measurements between two or more electrodes 202 disposed on a right side of the distal portion 106. Based on the impedance measurements, the external electrical components 110 can determine whether the distal portion 106 is positioned within the urethra of the patient at a location of the prostate 102.

As shown in FIG. 5B, the balloon 502 is in an expanded configuration in which the surface of the balloon 502 expands in an outward direction 504 relative to a longitudinal axis of the urethral catheter 108. For example and without limitation, based on determining that the distal portion 106 is positioned within the urethra of the patient at a location of the prostate 102, the external electrical components 110 can cause the balloon 502 to inflate. In various embodiments, the external electrical components 110 can cause the balloon 502 to inflate by actuating a pump included in the external electrical components 110. The pump can pump air or fluid through the conduit 304 and into the balloon 502. The inflation of the balloon 502 can press against the prostate 102 and can secure the distal portion 106 of the urethral catheter 108 at the location of the prostate 102.

While not shown, in various embodiments, the urethral catheter 108 includes a urinary cannula. For example and without limitation, the conduit 304 of any of FIGS. 3, 4, and/or 5A-B could include a urinary cannula in addition to (e.g., alongside) the wires 302. The urinary cannula could convey urine for drainage of the bladder. In patients with an enlarged prostate 102 that interferes with the drainage of urine from the bladder, the medical device 100 could inflate the balloon 502 when the external electrical components 110 determine that the distal portion 106 is positioned at the location of the prostate 102. Inflating the balloon 502 can relieve pressure of the prostate 102 upon the urethra to permit the drainage of the bladder. The determination of the tissue type can cause the balloon 502 to be inflated only when the distal portion 106 is positioned at the location of the prostate 102. The determination of the tissue type can avoid inflating the balloon 502 when the distal portion 106 is not positioned at the location of the prostate 102. Selectively inflating the balloon only when the distal portion 106 is positioned within the urethra at the location of the prostate 102 can improve the comfort of the urethral catheter 108 for the patient. Also, selectively inflating the balloon can avoid damaging portions of the urethra that could occur when the distal portion 106 is not positioned within the urethra at the location of the prostate 102.

While not shown, in various embodiments, the urethral catheter 108 includes one or more insertion depth markings along a length of the urethral catheter 108. For example and without limitation, the insertion depth markings could indicate a distance along the length relative to the tip 308 of the urethral catheter 108. The insertion depth markings can indicate, to a healthcare professional, a measurement of the inserted length of the urethral catheter 108 into the urethra of the patient. The insertion depth markings can aid the healthcare professional in estimating the position of the distal portion 106 of the urethral catheter 108 relative to the location of the prostate 102. The determination of tissue types can verify or correct the estimate of the position of the distal portion 106 (e.g., by determining whether the tissue type associated with the location of the distal portion 106 is a prostate tissue type or a non-prostate tissue type).

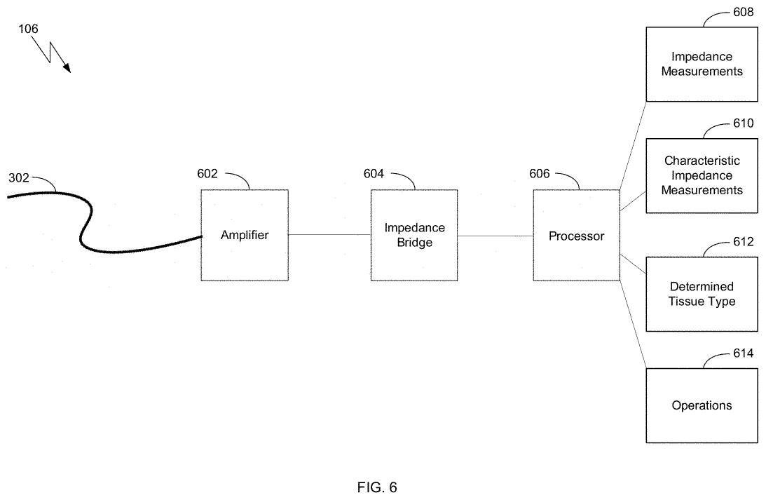

FIG. 6 is a more detailed illustration of the external electrical components of FIG. 2, according to various embodiments. As shown, the external electrical components 110 include wires 302, an amplifier 602, an impedance bridge 604, and a processor 606. The wires 302 conduct current at various frequencies between two or more electrodes 202 and the external electrical components 110. In various embodiments, the amplifier 602 is an analog interface amplifier that amplifies a supplied voltage and/or a return voltage while the wires 302 conduct current at various frequencies between the impedance bridge 604 and the two or more electrodes 202. In various embodiments, the impedance bridge 604 is an impedance load that the processor 606 measures to determine an impedance of a circuit including the impedance bridge 604, the amplifier 602, and the two or more electrodes 202. The processor 606 generates frequencies for a current that the wires 302 conduct between the impedance bridge 604 and the selected two or more electrodes 202.

While the wires 302 conduct current at various frequencies, the processor 606 records one or more impedance measurements 608 of the circuit including the at least two electrodes 202. The processor 606 compares the one or more impedance measurements 608 with characteristic tissue types 612 of respective one or more tissue types. Based on the one or more impedance measurements 608 and the characteristic tissue types 612, the processor 606 determines one or more tissue types 612 of tissue at the location associated with the distal portion 106 of the urethral catheter 108. For example and without limitation, based on the one or more impedance measurements 608 and the characteristic tissue types 612, the processor 606 can determine which tissue type is associated with characteristic impedance measurements 610 that are closest to the impedance measurements of the portion of tissue between at least two of the two or more electrodes 202. In various embodiments, the processor 606 can determine a Cole relaxation frequency of the portion of tissue based on the impedance measurements 608, and can compare the Cole relaxation frequency to one or more characteristic Cole relaxation frequencies of one or more tissue types. The Cole relaxation frequency corresponds to a frequency associated with a greatest impedance measurement 608 included in the one or more impedance measurements 608. In various embodiments, the Cole relaxation frequency is a frequency of a maximum normalized impedance measurement of the portion of tissue between at least two of the two or more electrodes 202. For example and without limitation, based on a Cole relaxation frequency below a threshold frequency (e.g., 10⁵ Hz), the processor 606 can determine that the portion of tissue between at least two of the two or more electrodes 202 is a non-tumor tissue type. Similarly, for example and without limitation, based on a Cole relaxation frequency above the threshold frequency, the processor 606 can determine that the portion of tissue between at least two of the two or more electrodes 202 is a tumor tissue type. As another example and without limitation, based on a Cole relaxation frequency below a threshold frequency, the processor 606 can determine that the portion of tissue between at least two of the two or more electrodes 202 is a prostate tissue type. Similarly, for example and without limitation, based on a Cole relaxation frequency above the threshold frequency, the processor 606 can determine that the portion of tissue between at least two of the two or more electrodes 202 is a non-prostate tissue type.

In some embodiments, the processor 606 determines two or more tissue types of tissue at the location associated with the distal portion 106 of the urethral catheter 108 based on impedance measurements respectively recorded by different electrode pairs of the two or more electrodes 202. For example and without limitation, the distal portion 106 can include a first electrode pair disposed at a first location along the length of the distal portion 106 and a second electrode pair disposed at a second location along the length of the distal portion 106. While the urethral catheter 108 is inserted into a urethra of a patient, the processor 606 can determine tissue types at the first location and the second location based on the respective impedance measurements associated with the first electrode pair and the second electrode pair. As another example and without limitation, the distal portion 106 can include a first electrode pair disposed on a left side of the distal portion 106 and a second electrode pair disposed on a right side of the distal portion 106. While the urethral catheter 108 is inserted into a urethra of a patient, the processor 606 can determine tissue types at the left side and the right side of the distal portion 106 based on the respective impedance measurements associated with the first electrode pair and the second electrode pair.

In various embodiments in which the medical device 100 includes an output component, the processor 606 performs one or more operations 614 to output an indication of the one or more tissue types at the location of the distal portion 106. In various embodiments and without limitation, the medical device 100 can display the one or more tissue types 612 at the location associated with the distal portion 106 using a visual output (e.g., a liquid crystal display (LCD), a light-emitting diode (LED) display to present a visual indication of the one or more tissue types 612 at the location associated with the distal portion 106, such as a light, symbol, text, graphic, or the like). In various embodiments and without limitation, the processor 606 can output a visual indication that the one or more tissue types 612 at the location associated with the distal portion 106 is a prostate tissue type. The visual indication can indicate whether or not the distal portion 106 of the urethral catheter 108 is positioned at the location of the prostate.

In various embodiments, the external electrical components 110 includes a wireless transmitter. For example and without limitation, the wireless transmitter can include a Bluetooth transmitter or a WiFi transmitter. The processor 606 can transmit, using the wireless transmitter, a wireless signal indicating the one or more tissue types at the location associated with the distal portion 106 of the urethral catheter 108. For example and without limitation, the wireless signal can be transmitted to a mobile device of a healthcare professional. Transmitting the wireless signal can cause the mobile device to output a visual indication or an audio indication of the determined tissue type 612.

In various embodiments in which the urethral catheter 108 includes a medical device tool, the processor 606 performs one or more operations 614 to control the medical device tool based on the one or more tissue types 612 at the location associated with the distal portion 106. For example and without limitation, in various embodiments in which the distal portion 106 includes a balloon, the processor 606 can perform operations 614 that include causing the balloon to inflate (e.g., by actuating a pump to inject air and/or water into the balloon). For example and without limitation, in various embodiments in which the urethral catheter 108 includes a therapeutic drug delivery tool, the processor 606 can perform operations 614 that include activating the therapeutic drug delivery tool to deliver one or more therapeutic drugs to the tissue at the location associated with the distal portion 106. For example and without limitation, in various embodiments in which the urethral catheter 108 includes an energy delivery tool, the processor 606 can perform operations 614 that include activating the energy delivery tool to deliver energy to the tissue at the location associated with the distal portion 106.

In various embodiments, the processor 606 can determine a confidence score of the one or more tissue types at the location associated with the distal portion 106. For example and without limitation, the processor 606 can determine a magnitude of difference between the impedance measurements 608 of the electrodes 202 that are associated with a prostate tissue type and the impedance measurements 608 of the electrodes 202 that are associated with a non-prostate tissue type. Alternatively or additionally, the processor 606 can determine a magnitude of difference between the impedance measurements 608 of the electrodes 202 that are associated with a cancer tissue type and the impedance measurements 608 of the electrodes 202 that are associated with a non-cancer tissue type. The confidence score can be based on (e.g., proportional to) the magnitude of difference between the different impedance measurements 608. As another example and without limitation, the processor 606 can determine a magnitude of the difference between the impedance measurements 608 for a set of electrodes 202 and the closest set of characteristic impedance measurements 610 of a tissue type. The confidence score can be based on (e.g., proportional to) the magnitude of the difference between the recorded impedance measurements 608 and the characteristic impedance measurements 600 of the tissue type. As yet another example and without limitation, the processor 606 can determine a signal-to-noise ratio of one or more of the impedance measurements 608. In various embodiments and without limitation, the processor 606 outputs the confidence score along with the determined tissue type 612. The confidence score can indicate to a healthcare professional a confidence of the determined tissue type 612 and/or a confidence of whether the distal portion 106 is positioned within the urethra at a location of the prostate 102.

In various embodiments, the processor 606 can measure one or more dimensions of tissue residing at the location associated with the distal portion 106. For example and without limitation, in embodiments including electrodes 202 positioned along a length of the distal portion 106, the processor 606 can selectively couple to respective pairs of adjacent electrodes 202 and can record a subset of impedance measurements 608 for each pair of electrodes 202. Based on the tissue types 612 determined for each subset of impedance measurements 608, the processor 606 can determine which pairs of electrodes 202 (e.g., which lengthwise portions of the distal portion 106) are in contact with prostate tissue and which pairs of electrodes 202 are not in contact with prostate tissue. Based on these determinations, the processor 606 can determine a length of the prostate 102 of the patient.

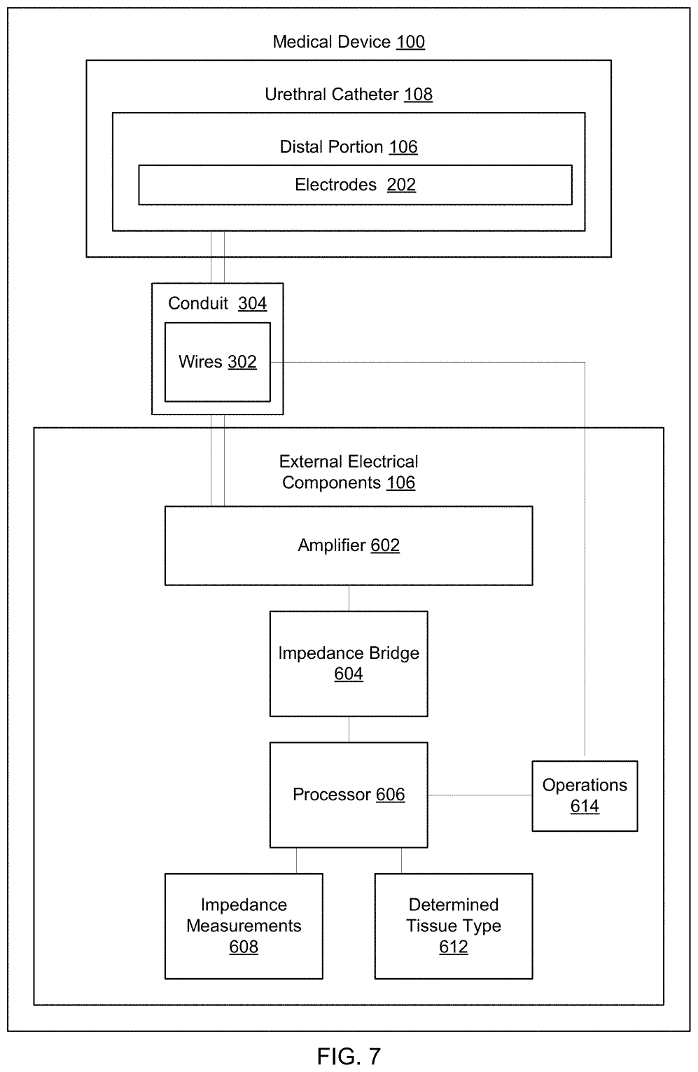

FIG. 7 is a more detailed illustration of the medical device 200 of FIG. 2, according to various embodiments. As shown, the medical device 200 includes a urethral catheter 108, a conduit 304, and external electrical components 110. As shown, the urethral catheter 108 includes a distal portion 106, and the distal portion 106 includes two or more electrodes 202 that are disposed on the distal portion 106. In some embodiments, the distal portion 106 includes a medical device tool, such as and without limitation, a balloon, a therapeutic drug delivery tool, an energy delivery tool, or a tissue sample extraction tool. In various embodiments, the distal portion 106 includes, without limitation, two or more medical device tools, which can be of one kind or of different kinds.

As shown, the conduit 304 includes wires 302 that couple the two or more electrodes 202 to the external electrical components 110. In various embodiments, without limitation, each of the two or more electrodes 202 is coupled to the external electrical components 110 by one wire 302 or by respective wires of a plurality of wires 302.

As shown, the external electrical components 110 include an amplifier 602, an impedance bridge 604, and a processor 606. The amplifier 602 amplifies a supplied voltage and/or a return voltage while the wires 302 conduct current at various frequencies between the impedance bridge 604 and the two or more electrodes 202. The impedance bridge 604 is an impedance load that the processor 606 measures to determine an impedance of a circuit including the impedance bridge 604, the amplifier 602, the wires 302, and the two or more electrodes 202. The processor 606 records, at various frequencies, one or more impedance measurements 608. The processor 606 compares the two or more impedance measurements 608 with characteristic impedance measurements 610 of respective one or more tissue types. Based on the one or more impedance measurements 608 with characteristic tissue types 612, the processor 606 determines one or more tissue types 612 at the location associated with the distal portion 106. In various embodiments and without limitation, the processor 606 determines the tissue type 612 indicated by the respective impedance measurements 608 based on a Cole relaxation frequency of a portion of tissue contacting the two or more electrodes 202. In various embodiments and without limitation, the processor 606 determines the tissue type 612 as areas of tumor tissue types and/or non-tumor tissue types. In various embodiments and without limitation, the processor 606 determines the tissue type 612 as prostate tissue types and/or non-prostate tissue types.

As shown, based on the one or more tissue types 612 at the location associated with the distal portion 106, the processor 606 performs one or more operations 614. In various embodiments and without limitation, if the tissue type determined at the location associated with the distal portion 106 is a prostate tissue type, the processor 606 performs an operation 614 of outputting a visual indication or an audio indication of the tissue type. In various embodiments and without limitation, the medical device 100 reports the one or more tissue types 612 at the location associated with the distal portion 106 to a user of the medical device 100. For example and without limitation, the medical device 100 can display the one or more tissue types 612 at the location associated with the distal portion 106 using a visual output (e.g., a liquid crystal display (LCD), a light-emitting diode (LED) display to present a visual indication of the one or more tissue types 612 at the location associated with the distal portion 106, such as a light, symbol, text, graphic, or the like). In various embodiments and without limitation, the visual indication can indicate the one or more tissue types 612 at the location associated with the distal portion 106 is a prostate tissue type. The visual indication can indicate whether or not the distal portion 106 of the urethral catheter 108 is positioned at the location of the prostate. The visual indication can indicate whether the tissue type of the prostate is a cancer tissue type or a non-cancer tissue type.

Alternatively or additionally, in various embodiments, the processor 606 performs one or more operations 614 associated with additional components associated with the distal portion 106 of the urethral catheter 108. In various embodiments and without limitation, if the tissue type determined at the location associated with the distal portion 106 is a prostate tissue type, the processor 606 performs an operation 614 of causing a balloon included in the distal portion 106 of the urethral catheter 108 to inflate. In various embodiments and without limitation, the urethral catheter 108 includes a therapeutic drug delivery tool, and the processor 606 performs an operation 614 of causing the therapeutic drug delivery tool to deliver one or more therapeutic drugs to tissue at the location associated with the distal portion 106. For example and without limitation, the processor 606 can cause one or more therapeutic drugs through one or more drug delivery conduits to and through the distal portion 106. In various embodiments and without limitation, the urethral catheter 108 includes an energy delivery tool, and the processor 606 performs an operation 614 of causing the energy delivery tool to deliver energy to tissue at the location associated with the distal portion 106. For example and without limitation, the processor 606 can cause current to be conducted through wires in the conduit 304 to and through the distal portion 106.

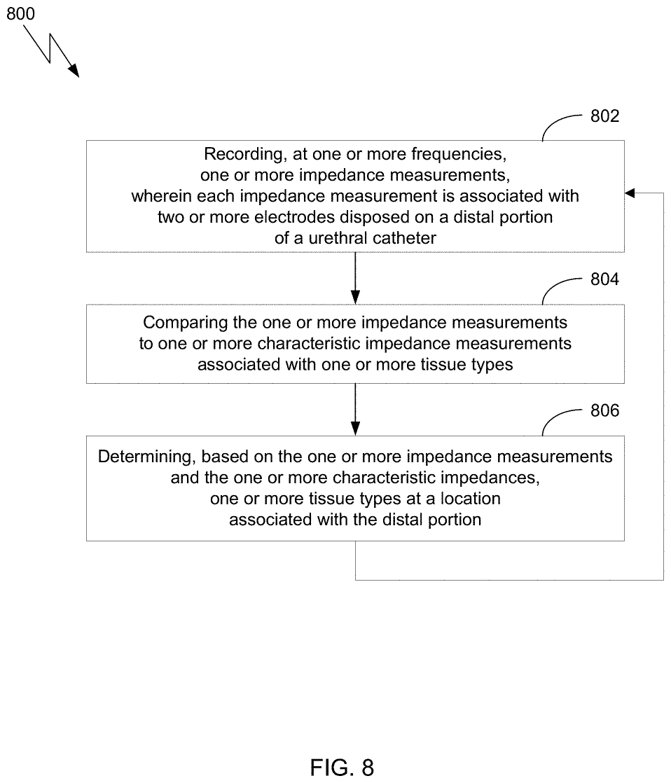

FIG. 8 is a flow diagram of method steps for determining one or more tissue types at a location associated with a distal portion of a urethral catheter, according to various embodiments. Although the method steps are described in conjunction with the systems of FIGS. 1-7, persons skilled in the art will understand that any system configured to perform the method steps, in any order, falls within the scope of the present invention.

As shown, a method 800 begins at step 802, where a processor 606 records, at one or more frequencies, one or more impedance measurements, wherein each impedance measurement is associated with two or more electrodes disposed on a distal portion of a urethral catheter. In various embodiments and without limitation, the processor 606 determines a Cole relaxation frequency of tissue between at least two of the two or more electrodes disposed on the distal portion of the urethral catheter (e.g., without limitation, as a frequency of a maximum normalized impedance measurement of the tissue between at least two of the two or more electrodes).

At step 804, the processor compares the one or more impedance measurements to one or more characteristic impedances associated with one or more tissue types. For example and without limitation, the processor can compare the impedance measurements with a first set of one or more characteristic impedance measurements of a non-tumor tissue type and a second set of one or more characteristic impedance measurements of a tumor tissue type.

At step 806, the processor determines, based on the one or more impedance measurements and the one or more characteristic impedances, one or more tissue types at a location associated with the distal portion. In various embodiments and without limitation, the processor determines the tissue type that classifies the tissue as one of a tumor tissue type or a non-tumor tissue type. In various embodiments and without limitation, the processor determines whether the tissue type at the location associated with the distal portion of the urethral catheter matches an expected tissue type of the prostate. The method can return to step 802 to record additional impedance measurements and to determine a second or updated tissue type.

In sum, the disclosed medical device measures the impedance of tissue in a location associated with a distal portion of a urethral catheter. The medical device determines the tissue type based on impedance measurements associated with two or more electrodes disposed on the distal portion of the catheter. The disclosed approach advantageously results in the medical device determining the tissue types of tissue associated with the location of the distal portion of the urethral catheter.

At least one technical advantage of the disclosed medical device relative to the prior art is that the disclosed medical device is able to automatically and accurately determine the tissue type at a location associated with the distal portion of the urethral catheter. For example, the disclosed urethral catheter can accurately determine whether the tissue type associated with a prostate is a cancerous tissue type or a non-cancerous tissue type. Determining the tissue type associated with a prostate with high accuracy can advantageously reduce the incidence of false positive testing outcomes and reduce the occurrence of unnecessary medical procedures. Determining the tissue type associated with a prostate with high accuracy also can advantageously reduce the incidence of false negative testing outcomes and enable effective cancer treatment at early stages and with better prognoses. In addition, the disclosed medical device can automatically and accurately determine whether the tissue type at a location associated with the distal portion of the urethral catheter matches a tissue type associated with a prostate. Such determinations can improve overall confidence in the test results. Likewise, automatically and accurately determining that the tissue type at a location associated distal portion of the urethral catheter does not match the tissue type associated with a prostate can reduce incorrect test results and give a healthcare provider an opportunity to reposition the urethral catheter. These technical advantages provide one or more technological advancements over prior art designs and approaches.

1. In various embodiments, a medical device comprises a urethral catheter including, a distal portion, and two or more electrodes disposed on the distal portion; an impedance bridge coupled to the two or more electrodes; and a processor coupled to the impedance bridge.

2. The medical device of clause 1, wherein each of the two or more electrodes is located at a respective location along a length of the distal portion.

3. The medical device of clauses 1 or 2, wherein at least one electrode of the two or more electrodes is located on a first side of the distal portion, and at least another one electrode of the two or more electrodes is located on a second side of the distal portion.

4. The medical device of any of clauses 1-3, further comprising an activation button coupled to the processor.

5. The medical device of any of clauses 1-4, further comprising a display coupled to the processor.

6. The medical device of any of clauses 1-5, further comprising a wireless transmitter coupled to the processor.

7. The medical device of any of clauses 1-6, wherein the processor selectively couples the impedance bridge to at least two electrodes of the two or more electrodes.

8. The medical device of any of clauses 1-7, wherein the distal portion includes at least one of a balloon or a urinary cannula.

9. The medical device of any of clauses 1-8, wherein one or more insertion depth markings are disposed along a length of the urethral catheter.

10. In various embodiments, a computer-implemented method for determining one or more tissue types at a location associated with a distal portion of a urethral catheter comprises recording, at one or more frequencies, one or more impedance measurements, wherein each impedance measurement is associated with two or more electrodes disposed on the distal portion of the urethral catheter; comparing the one or more impedance measurements to one or more characteristic impedances associated with one or more tissue types; and determining, based on the one or more impedance measurements and the one or more characteristic impedances, one or more tissue types at a location associated with the distal portion.

11. The computer-implemented method of clause 10, wherein the one or more tissue types include at least one of a cancer tissue type, a non-cancer tissue type, a prostate tissue type, or a non-prostate tissue type.

12. The computer-implemented method of clauses 10 or 11, wherein the one or more impedance measurements are recorded in response to an activation associated with an activation button.

13. The computer-implemented method of any of clauses 10-12, wherein the one or more impedance measurements include a first impedance measurement associated with a first subset of the two or more electrodes, and a second impedance measurement associated with a second subset of the two or more electrodes.

14. The computer-implemented method of any of clauses 10-13, wherein the first subset of the two or more electrodes is associated with a first location along the distal portion, and the second subset of the two or more electrodes is associated with a second location along the distal portion.

15. The computer-implemented method of any of clauses 10-14, wherein the first subset of the two or more electrodes is associated with a first side of the distal portion, and the second subset of the two or more electrodes is associated with a second side of the distal portion.

16. The computer-implemented method of any of clauses 10-15, further comprising measuring one or more dimensions of tissue residing at the location associated with the distal portion.

17. The computer-implemented method of any of clauses 10-16, further comprising outputting at least one of a visual indication or an audio indication of the one or more tissue types at the location associated with the distal portion.

18. The computer-implemented method of any of clauses 10-17, further comprising determining a confidence score associated with the one or more tissue types at the location associated with the distal portion.

19. The computer-implemented method of any of clauses 10-18, further comprising transmitting, via a wireless transmitter, a wireless signal indicating the one or more tissue types at the location associated with the distal portion.

20. The computer-implemented method of any of clauses 10-19, further comprising causing a balloon included in the distal portion of the urethral catheter to inflate.

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A medical device, comprising:
a urethral catheter including,
a distal portion, and
two or more electrodes disposed on the distal portion;
an impedance bridge coupled to the two or more electrodes; and
a processor coupled to the impedance bridge.

2. The medical device of claim 1, wherein each of the two or more electrodes is located at a respective location along a length of the distal portion.

3. The medical device of claim 1, wherein at least one electrode of the two or more electrodes is located on a first side of the distal portion, and at least another one electrode of the two or more electrodes is located on a second side of the distal portion.

4. The medical device of claim 1, further comprising an activation button coupled to the processor.

5. The medical device of claim 1, further comprising a display coupled to the processor.

6. The medical device of claim 1, further comprising a wireless transmitter coupled to the processor.

7. The medical device of claim 1, wherein the processor selectively couples the impedance bridge to at least two electrodes of the two or more electrodes.

8. The medical device of claim 1, wherein the distal portion includes at least one of a balloon or a urinary cannula.

9. The medical device of claim 1, wherein one or more insertion depth markings are disposed along a length of the urethral catheter.

10. A computer-implemented method for determining one or more tissue types at a location associated with a distal portion of a urethral catheter, the computer-implemented method comprising:
recording, at one or more frequencies, one or more impedance measurements, wherein each impedance measurement is associated with two or more electrodes disposed on the distal portion of the urethral catheter;
comparing the one or more impedance measurements to one or more characteristic impedances associated with one or more tissue types; and
determining, based on the one or more impedance measurements and the one or more characteristic impedances, one or more tissue types at a location associated with the distal portion.

11. The computer-implemented method of claim 10, wherein the one or more tissue types include at least one of a cancer tissue type, a non-cancer tissue type, a prostate tissue type, or a non-prostate tissue type.

12. The computer-implemented method of claim 10, wherein the one or more impedance measurements are recorded in response to an activation associated with an activation button.

13. The computer-implemented method of claim 10, wherein the one or more impedance measurements include a first impedance measurement associated with a first subset of the two or more electrodes, and a second impedance measurement associated with a second subset of the two or more electrodes.

14. The computer-implemented method of claim 13, wherein the first subset of the two or more electrodes is associated with a first location along the distal portion, and the second subset of the two or more electrodes is associated with a second location along the distal portion.

15. The computer-implemented method of claim 13, wherein the first subset of the two or more electrodes is associated with a first side of the distal portion, and the second subset of the two or more electrodes is associated with a second side of the distal portion.

16. The computer-implemented method of claim 10, further comprising measuring one or more dimensions of tissue residing at the location associated with the distal portion.

17. The computer-implemented method of claim 10, further comprising outputting at least one of a visual indication or an audio indication of the one or more tissue types at the location associated with the distal portion.

18. The computer-implemented method of claim 10, further comprising determining a confidence score associated with the one or more tissue types at the location associated with the distal portion.

19. The computer-implemented method of claim 10, further comprising transmitting, via a wireless transmitter, a wireless signal indicating the one or more tissue types at the location associated with the distal portion.

20. The computer-implemented method of claim 10, further comprising causing a balloon included in the distal portion of the urethral catheter to inflate.
